(19) United States
(12) Patent Application Publication
(10) Pub. No.: US 2020/0330132 A1
(43) Pub. Date: Oct. 22, 2020

(54) GROWTH AND MOTION SPARING TETHERS AND BONE ANCHOR IMPLANTS FOR THE TREATMENT AND CORRECTION OF SPINE DEFORMITIES

(71) Applicant: Harsh Grewal, Gladwyne, PA (US)

(72) Inventor: Harsh Grewal, Gladwyne, PA (US)

(21) Appl. No.: 16/388,040

(22) Filed: Apr. 18, 2019

(51) Int. Cl. A61B 17/70; A61F 2/46; A61F 2/44

(52) U.S. Cl. CPC A61B 17/7022; A61F 2/4611; A61B 17/86; A61B 17/7035; A61F 2310/00023; A61F 2/44

(57) ABSTRACT

Tethers, devices and bone anchor implants are provided for the correction and treatment of spine deformities including scoliosis.

### FIELD OF THE INVENTION

This invention relates to tethers and bone anchor implants that are minimally invasive, growth and motion sparing treatments that may utilize growth modulation without rigid bony fusion of the spine for correction of deformities of the spine such as scoliosis.

### BACKGROUND OF THE INVENTION

Deformities of the spine may result in abnormal curvature of the spine (scoliosis and kyphosis and various combinations) and are the result of a number of causes, including congenital deformities, neuromuscular diseases, as well as idiopathic and early onset scoliosis.

Scoliosis, an abnormal curvature of the spine, affects 2-3% of the population, or an estimated 7 million people in the United States, and there is no cure (National Scoliosis Foundation). In 2011, an estimated 442,900 office visits, 133,300 hospital visits, and 17,500 emergency room visits were made by children with scoliosis. The average cost of a hospital stay for a child with scoliosis in 2011 was $92,000—over five times the US national average of $17,500 (Health Care Utilization Project, AHRQ, 2011). An estimated 30,000 to 40,000 of these patients will undergo spinal fusion surgery for scoliosis in the US because of failure of current non-surgical treatments, such as bracing. The health care cost in the pediatric population for scoliosis surgery is second only to surgical care of appendicitis in the US. In 2009 there were more than 3600 hospital discharges for spinal surgery for AIS, and the total costs of ˜$514 million ranked 2nd only to appendicitis in children 10 to 17 years of age (AHRQ).

The patient population that will use the present invention are children with idiopathic scoliosis who are skeletally immature and are still growing with progression of their curve. There is no specific age range for the present invention (these children are usually adolescents), but the patient selection is based on the degree of skeletal immaturity (Risser grade or Sanders stage from radiographs are used to quantify the maturity of the skeleton). Current suggested indications for utilizing a non-fusion treatment (there are no FDA approved systems for this indication) as an alternative to bracing in idiopathic scoliosis (adolescent or juvenile) are age >10 years old with remaining spine growth, i.e. Risser grade 0-2, Sanders stage ≤4 and a thoracic curve 35° to 60° which is flexible below 30° on bending films.

The current treatment for skeletally immature patients with idiopathic scoliosis and moderate curves (20-40°) is observation and bracing. Bracing does not always work and has poor compliance in young patients secondary to psychosocial and body image considerations. Patients who fail bracing and whose curves keep progressing are treated with spinal fusion. Fusion of the spine may be performed from an anterior or posterior or combined approach with insertion screws, bone grafts and rigid rods. These techniques result in significant limitation of motion of the spine as well as limitation of potential growth of the spine when performed in the skeletally immature spine. In addition, these fusion techniques, especially posterior spinal fusions require large incisions and consequently are painful, have a potentially large scar that may not be cosmetically satisfactory, and have significant infectious complications as well as significant potential blood loss from surgery. Additionally, there are risks of mechanical failure of the implanted constructs and non-fusion, as well as damage and degeneration to the adjacent intervertebral discs.

Thus growth and motion sparing treatments for spine deformities, especially for early onset and idiopathic scoliosis that avoid spinal fusion are needed. A number of alternate techniques have been described including growing rods, vertically expandable prosthetic titanium rib constructs, growth guidance procedure (SHILLA™) and vertebral body stapling. All these techniques appear to have drawbacks, including multiple operations (titanium rib, growing rod), unintended auto-fusion of the spine (growing rods) and, re operations (SHILLA™), and limited indications such as in vertebral body stapling.

A recently introduced method is anterior vertebral body tethering. This technique has been studied in animal models. Newton et al have demonstrated the ability of a unilateral tether to induce deformity in a bovine model. Braun et al have demonstrated this in a goat model. Clinical experience in humans is limited with a single published case report (Crawford et al) and two case series from a single center (Samdani et al). This technique is potentially motion and growth sparing. This method however utilizes a staple and a screw to be inserted into the vertebral body requiring sacrifice of the blood vessels as well as requiring multiple steps for the insertion (Methods and techniques for spinal surgery WO 2014143862 A1). In addition, a vertebral body screw in these techniques potentially could enter the spinal canal and cause spinal cord injury if the trajectory is in a posterior direction; if the screw is in an anterior trajectory there could be injury to the anteriorly located aorta; if the screw breaches the contralateral vertebral body cortex it could potentially result in injury to the aorta with immediate or delayed bleeding as a result of a pseudo-aneurysm of the aorta. These are potentially life and limb threatening devastating injuries. In addition, the currently available techniques for application of a vertebral body tether require extensive dissection of all the segmental spinal and vertebral blood supply. The sacrifice of all these blood vessels puts the spinal cord at a potential for ischemic injury. In addition, there is risk of bleeding during the control and dissection for exposure of the anterior spine and blood vessels for preparation of previously described implants, screws and tethers.

Thus there is a need for tethers, devices and bone anchor implants that are minimally invasive, that are growth and motion sparing treatments that avoid fusion of the spine for deformities of the spine and that can be performed using minimally invasive techniques while minimizing risks to the aorta and spinal cord. In addition, there is a need for tethers, devices and bone anchor implants that can spare the segmental blood supply of the spine and spinal cord, minimize the need for extensive intra-thoracic surgical dissection and sacrifice of normal blood supply, and mitigate the need for placing a screws in the vertebral body that could injure the spinal cord or aorta.

The relevant prior art comprises:
Braun J T, Ogilvie J W, Akyuz E, et al. Creation of an experimental idiopathic type scoliosis in an immature goat model using a flexible posterior asymmetric tether. Spine (Phila Pa. 1976) 2006; 31:1410-4.
Newton P O, Fricka K B, Lee S S, et al. Asymmetrical flexible tethering of spine growth in an immature bovine model. Spine (Phila Pa. 1976) 2002; 27:689-93.
Newton P O, Farnsworth C L, Faro F D, et al. Spinal growth modulation with an anterolateral flexible tether in an immature bovine model: disc health and motion preservation. Spine (Phila Pa. 1976) 2008; 33:724-33.
Crawford C H, 3rd, Lenke L G. Growth modulation by means of anterior tethering resulting in progressive correction of juvenile idiopathic scoliosis: a case report. J Bone Joint Surg Am 2010; 92:202-9.
Betz R R, Ranade A, Samdani A F, et al. Vertebral body stapling: a fusionless treatment option for a growing child with moderate idiopathic scoliosis. Spine (Phila Pa. 1976) 2010; 35:169-76.
Braun J T, Ogilvie J W, Akyuz E, et al. Experimental scoliosis in an immature goat model: a method that creates idiopathic type deformity with minimal violation of the spinal elements along the curve. Spine (Phila Pa. 1976) 2003; 28:2198-203.
Samdani A F, Ames R J, Kimball J S, Pahys J M, Grewal H, Pelletier G J, Betz R R Anterior vertebral body tethering for immature adolescent idiopathic scoliosis: one-year results on the first 32 patients. Eur Spine J. 2014 Dec. 16.
Samdani A F, Ames R J, Kimball J S, Pahys J M, Grewal H, Pelletier G J, Betz R R Anterior vertebral body tethering for idiopathic scoliosis: two-year results. Spine (Phila Pa. 1976). 2014 Sep. 15; 39(20):1688-93

### BRIEF DESCRIPTION OF THE INVENTION

Tethers, devices and bone anchor implants are provided for the correction and treatment of spine deformities including scoliosis. These tethers, devices and bone anchor implants are growth and motion sparing treatments that may utilize growth modulation without rigid bony fusion of the spine, especially when utilized in the immature and growing spine, such as early onset scoliosis and adolescent idiopathic scoliosis.

In an exemplary embodiment, a minimally invasive thoracoscopic technique is disclosed in which, under endoscopic and fluoroscopic guidance, the tethers and bone anchor implants of the present invention are inserted and fixed to the anterior or anterior-lateral aspect of the vertebral bodies of the deformed spine segments. The tether or tethers attach to the bone anchor implants thus tethering the vertebral bodies of the spine in the deformed segment of the spine without rigid bony fusion. The tethers, bone anchoring implants and other related devices may be inserted into multiple adjacent vertebral bodies of the spine usually spanning the length of the deformity on the convex side of the deformity, thus tethering the convex aspect of the deformity. The tethering of the convex side results in progressive correction of the curve and deformity resulting from this curve, as the untethered concave side grows proportionally faster than the tethered convex side of the deformed spine. These described tethers, bone anchoring devices and implants in this exemplary embodiment uniquely allow for conservation and preservation of the segmental blood supply of the spine and spinal cord as well as avoiding damage to the intervertebral disc, thus mitigating any damaging effects of the treatment to the spine and spinal cord as well as to the growth and motion of the spine. The present invention allows the tether to be anchored to the vertebral body without compromising the blood supply to the spinal cord. In addition, the present invention does not require the coagulation, interruption, mobilization or damage to this segmental blood supply thus additionally making the minimally invasive method less complex, safer and with less risk of bleeding.

In one embodiment a flexible biologically compatible cable, ribbon or tether is attached sequentially to the implanted anchoring devices in multiple vertebral bodies, tethering the vertebral bodies to the ones above and below them along the whole length of the deformed spine segment. In one exemplary embodiment of this invention utilizing these implants and devices, the flexible biologically compatible cable, ribbon or tether may be sequentially tightened between each segment and fixed in place to the implant resulting in immediate partial correction of the deformity. In addition, and significantly because there is no fusion of the spine, as well as sparing of the segmental spinal cord blood supply as well as preventing injury to the intervertebral disc, if this embodiment is used in a skeletally immature spine of children with early onset scoliosis and adolescents with idiopathic scoliosis, further growth of the unconstrained contralateral (concave) aspect of the spine allows for continued correction (and periodic adjustment) until skeletal growth is completed.

### SUMMARY OF THE INVENTION

This invention describes methods, tethers, devices and bone anchor implants that are minimally invasive and which overcome many of the deficiencies in the prior art.

These tethers, devices and bone anchor implants are unique and provide various embodiments that allow the correction of spine deformities especially in the growing spine of a child without rigid bony fusion. This invention allows for the correction of curvature of the spine by implanting bone anchors in multiple adjacent vertebral bodies of the lateral aspect of the convex side of the deformed spine that then are attached to a tether or tethers thus tethering the convex aspect of the spine thus allowing the contralateral untethered concave aspect of the spine to grow faster and thus correct the deformity by growth modulation. In another exemplary embodiment these implants and technique can achieve immediate correction by tightening the tether sequentially between two adjacent anchors, after all the vertebral bodies have had the bone anchor implants placed over the entire span of the convexity of the length of the deformity. The size and shape of the tether and bone anchoring implants as well as the inserting device allow implantation using a minimally invasive thoracoscopic and fluoroscopic technique utilizing valved trocars that allow carbon dioxide insufflation thus avoiding large incisions and improving visualization of the spine and reducing the time taken for the operative procedure.

### BRIEF DESCRIPTION OF THE DRAWINGS

FIG. 1 depicts an exploded perspective view of a first embodiment of the present invention, showing an anchor having four tines for engaging the vertebral body. as well as a locking collar and set-screw for securing a tether or cable to the anchor.

FIG. 2 depicts an assembled perspective view of the first embodiment of the present invention.

FIG. 3 depicts section perspective view of the first embodiment of the present invention.

FIG. 4 depicts a perspective view of the human spinal column, showing three anchors according to the first embodiment of the present invention in place on three vertebral bodies, and interconnected by a tether or cable.

FIG. 5 depicts a perspective view of a second embodiment of the present invention, showing an anchor having four tines for engaging the vertebral body. as well as a set-screw for securing a tether or cable to the anchor.

FIG. 6 depicts a perspective view of a third embodiment of the present invention, showing an anchor having two tines for engaging the vertebral body.

FIG. 7 depicts an exploded plan view of a fourth embodiment of the present invention, showing a screw having a cam.

FIG. 8 depicts a perspective view of a fifth embodiment of the present invention.

FIG. 9 depicts another perspective view of the fifth embodiment of the present invention.

### DETAILED DESCRIPTION OF THE INVENTION

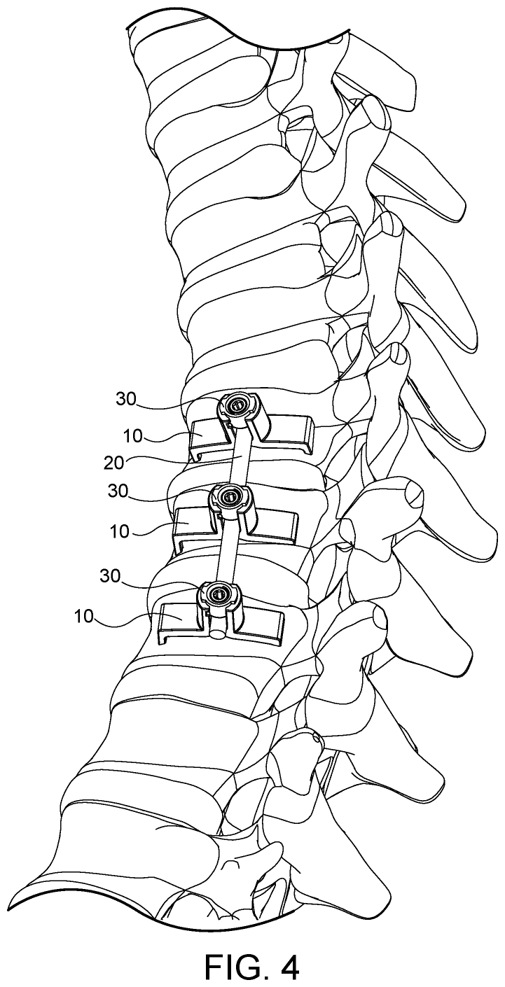

In a preferred embodiment, as shown in perspective view in FIG. 4, the bone anchor implant 10 of the present invention is implanted and anchors to the lateral aspect of the bone of the vertebral body while minimizing dissection of the overlying tissue and preserving the segmental blood supply of the spine. The several anchors 10 are joined together by tether 20, which is locked in place by locking caps 30.

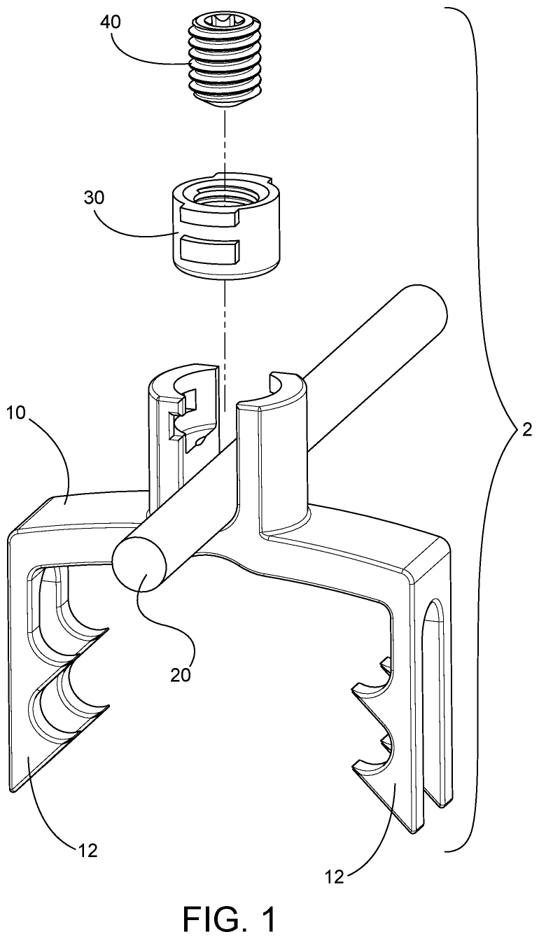

As depicted in FIG. 1, the bone anchor implant assembly 2 is comprised on anchor 10 that has four tines 12 shaped to allow them to span the segmental blood vessels and therefore on insertion, do not need these vessels and the overlying tissue such as the pleura to be dissected and coagulated. In addition, anchor 10 is adapted to receive locking cap 30, and it, in turn, receives set screw 40. Together locking cap 30 and set screw 40 retain tether in 20 in locked relation to anchor 10.

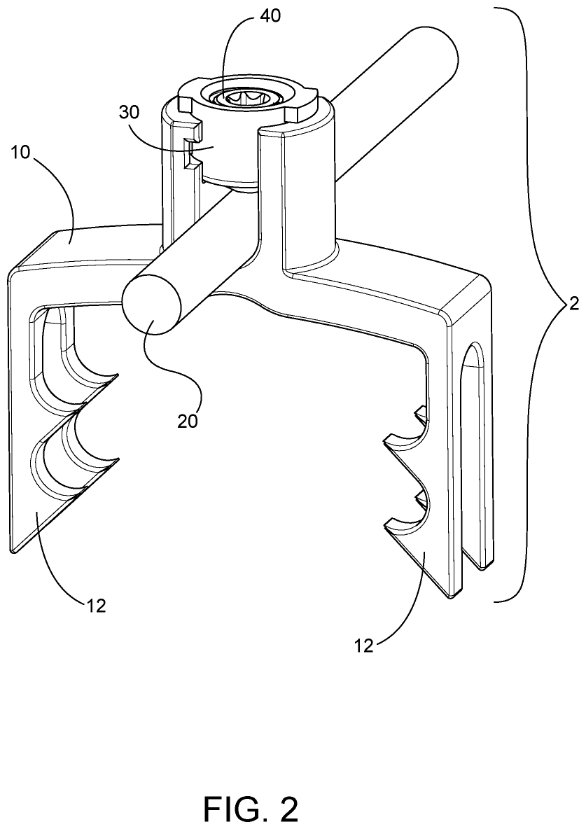

FIG. 2 further depicts implant assembly 2 with tether 20 locked in place by set screw 40.

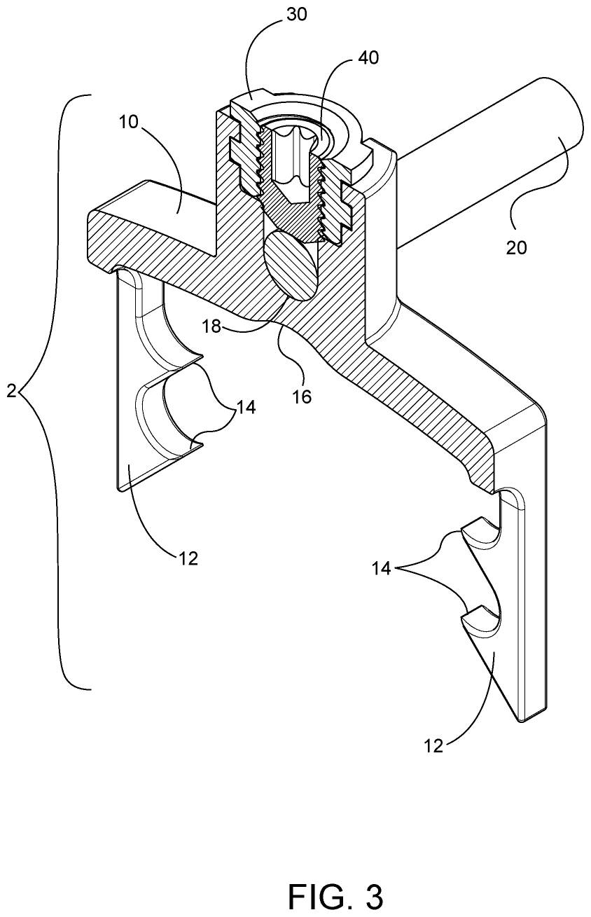

FIG. 3 further depicts implant assembly 2 in cross section, showing barbs 14, and groove 16 on the underside of the anchor 10 between the tines 12 that accommodates the segmental blood vessels.

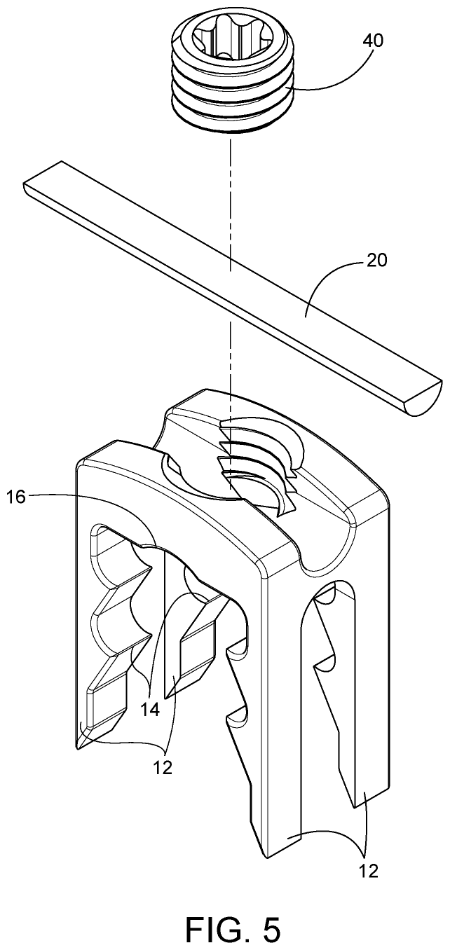

FIG. 5 depicts an alternative embodiment in which tether 20 may optionally have a semicircular cross-section. Set screw 40 is directly inserted into the body of the implant assembly which is threaded to accept it.

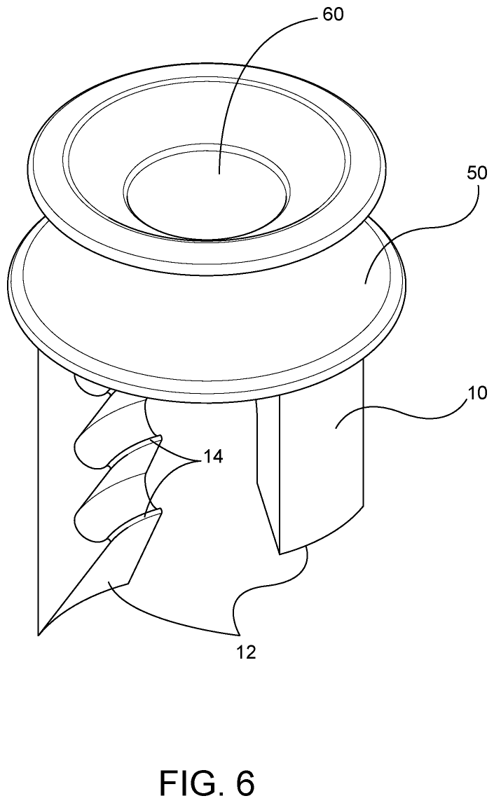

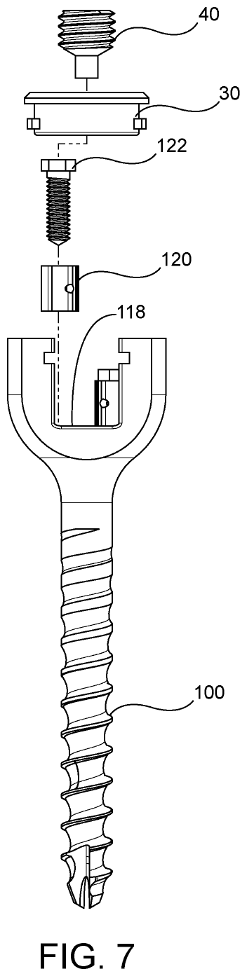

FIG. 6 depicts another alternative embodiment of the present invention. Tines 12 having barbs 14 engage the vertebral body. Outer channel 50 accommodates blood vessels routed around the anchor 10. Inner channel 60 is adapted to receive a screw, particularly the screw shown in FIG. 7.

FIG. 7 depicts a bicortical screw 100 having tether-receiving groove 118 in its head. Located within groove 118 is a pair of opposing eccentric cams 120, retained in place by cam screws 122. The head of screw 100 is adapted to receive locking cap 30 and set screw 40. In use, a tether 20 is placed between cams 120 and tensioned. Cams 120 allow one-way motion and grip tether 20. Once proper tension is achieved, set screw 40 is tightened to lock tether 20 in place.

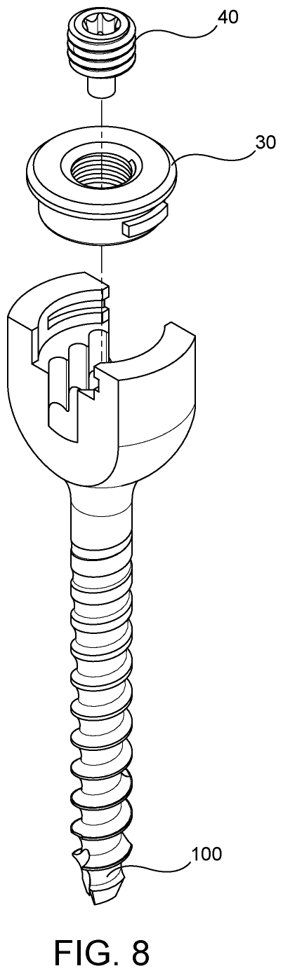

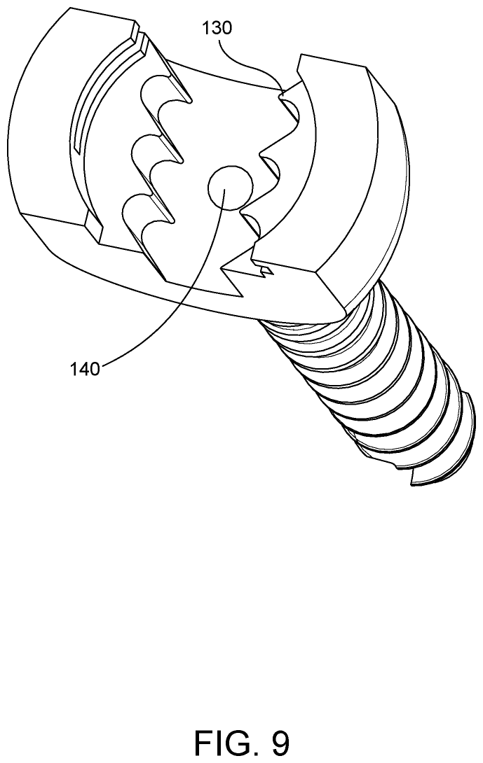

FIGS. 8 and 9 depict another embodiment of screw 100, in which a set of ridges 130 grip tether 20 (not shown). Locking cap 30 and set screw 40 then serve to retain and lock tether 20 in place. Central passage 140 allows the passage of a guide wire or similar instrument through the axis of the screw.

In every embodiment, the anchors 10 and the inserted screws 100 are placed to avoid the blood vessels as well as to avoid injury to the endplates and discs of the vertebral bodies that the bone anchor implant are being placed.

The tether 20 is made of bio-compatible cable that may be metallic or non-metallic. The material of the tether for example may be nonabsorbable or bioabsorbable. Portions of the tether 20 for example, may be rigid while some portions may be flexible or the tether or tethers 20 may be entirely flexible. The tether-receiving groove 18 (or 118) in each bone anchor implant is aligned with the similar tether-receiving groove 18 (or 118) in the adjacent bone anchor implant above and below, and thus the tether is attached to multiple bone anchor implants. As depicted, the groove has threads above the space for the tether to receive a set screw 40.

An inserting device (not shown) has a sleeve of metal or plastic that slides over the barbed tines allowing safe passage into the chest cavity through a valved endoscopic trocar, thus allowing easy passage through the valves, as well as protecting the lung and blood vessels from sharp edges as the bone anchor implant enters the hemithorax under thoracoscopic control and visualization. The inserting device also may be provided with two or more grooves or channels that allow the screws and screw driver to be inserted after the bone anchor implant has been placed into the vertebral body.

The bone anchor implants of the present invention are placed sequentially into the lateral aspect of the vertebral body through valved endoscopic trocars that are placed in the intercostal space under thoracoscopic visualization. Using the present invention the bone anchor implants can be placed safely without sacrificing soft tissue and blood vessels. Additionally, biplanar fluoroscopy can be utilized to ensure correct placement in each vertebral body. After insertion of all the bone anchor implants into the vertebral bodies spanning the deformity and confirming accurate placement utilizing thoracoscopy and fluoroscopy the tether is inserted into the chest cavity through the previously placed valved trocars again under thoracoscopic visualization. The tether is sequentially placed into the receiving groove or recess in this embodiment in each bone anchor implant. In one embodiment after the tether is placed in all the bone anchor implants the tether is locked or tightened into place, using, for example, a set screw or locking screw, in the most cephalad bone anchor implant. Having locked or fastened the tether to the most cephalad bone anchor implant, the tether is tightened and all the slack is removed and the next set screw or locking screw is placed thus sequentially fastening the tether. In another embodiment the tether may first be fastened in the middle bone anchor implant, which in this example may be at the apex of the deformity of the spine, and subsequently the tether is tightened and locked or screwed into place above and below this. After all the bone anchor implants have received the tether and the tether or tethers have been tightened sequentially under thoracoscopic and or fluoroscopic visualization the excess tether above and below the most cephalad and most caudad bone anchor implant is cut and the procedure is complete.

The present invention allows for conservation and preservation of the segmental blood supply of the spine and spinal cord as well as avoiding damage to the intervertebral disc, thus mitigating any damaging effects of the treatment to the spine and spinal cord as well as to the growth and motion of the spine. All other prior art devices such as screws for anterior vertebral body tethering require coagulation and interruption of the segmental blood supply of the spine and spinal cord, thus potentially risking injury from ischemia to the spine, discs and spinal cord. The present invention does not require the coagulation, interruption, mobilization or damage to this segmental blood supply, thus additionally making the minimally invasive method less complex, safer and with less risk of bleeding. It is less complex because there is no need to interfere with the segmental blood vessels that are branches from the aorta and can rapidly bleed if damaged without prior control.

Since the device of the present invention does not have to be inserted through the entire vertebral body and does not require multiple steps for insertion (e.g, does not require placement of a staple followed by a tap under fluoroscopy, as needed by a bicortical screw, as needed by the current methods it makes the operative procedure much simpler, quicker and safer.

In addition, since the device is uniquely designed to be inserted through currently available valved endoscopic ports the procedure can be performed thoracoscopically without any large thoracic or lumbar incision for implanting in the thoracic and lumbar spine.

While the present invention has been described in its preferred embodiments, it is to be understood that the words which have been used are words of description rather than of limitation and that changes may be made within the purview of the appended claims without departing from the true scope and spirit of the invention in its broader aspects. Rather, various modifications may he made in the details within the scope and range of equivalents of the claims and without departing from the spirit of the invention. The inventor further requires that the scope accorded their claims be in accordance with the broadest possible construction available under the law as it exists on the date of filing hereof (and of the application from which this application obtains priority, if any) and that no narrowing of the scope of the appended claims be allowed due to subsequent changes in the law, as such a narrowing would constitute an ex post facto adjudication, and a taking without due process or just compensation.

I claim as my invention:

1. A system for correction of spinal deformities comprising:
(a) two or more bone anchor implants each having at least two tines, each tine having barbs on at least one surface thereof to engage the bone of a vertebral body; each implant having at least two recesses adapted to accept screws for securing the implant to a vertebral body; each bone anchor implant further having a groove or channel adapted to allow unrestricted blood flow through the vertebral vessels; each implant further having a groove for accepting a tether, the groove having a locking device for securing the tether to bone anchor implant; and
(b) a tether for connecting the bone anchor implants one to another.

2. The system of claim 1 wherein the bone anchor implant has two tines opposed to each other, having barbs on the opposed surfaces thereof.

3. The system of claim 1 wherein the bone anchor implant has four tines, arranged in two opposed pairs, each opposed pair having barbs on the opposed surfaces thereof, wherein the bone anchor implant has one recess adapted to accept a screw positioned between each pair of adjacent tines.

4. The system of claim 1 wherein the bone anchor implant has two tines, offset from each other, each having barbs of the surface thereof to engage the bone of a vertebral body, wherein the bone anchor implant has a recess adapted to accept a screw positioned opposite each tine.

5. The system of claim 1 wherein the locking device is a set screw.

6. The system of claim 1 further comprising at least two screws for each bone anchor implant, for securing the implant to a vertebral body.

7. The system of claim 6 wherein the screws are bicortical screws.

8. The system of claim 1 wherein the tether is rigid.

9. The system of claim 1 wherein the tether is flexible.

10. The system of claim 1 wherein some portions of the tether are rigid, and other portions are flexible.

11. The system of claim 1 wherein the tether is bioabsorbable.

12. A tool for the emplacement of a bone anchor implant having tines, comprising a cannula adapted to enclose the tines of the implant, and enclosing a grasper/inserter that engages the implant, which further encloses a screw guide for guiding the insertion, removal, and adjustment of one or more screws that secure the implant to a vertebral body, and one or more screws that secure a tether to the implant.
